(19) United States
(12) Patent Application Publication
(10) Pub. No.: US 2007/0003503 A1
(43) Pub. Date: Jan. 4, 2007

(54) Tissue enhancement implant and method

(75) Inventor: Vladimir Sabetsky, Stockholm (SE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: The Technology Development Co., Ltd.

(21) Appl. No.: 11/110,282

(22) Filed: Apr. 20, 2005

Related U.S. Application Data
(60) Provisional application No. 60/563,474, filed on Apr. 20, 2004.

(51) Int. Cl. A61K 39/08; A61K 8/73

(52) U.S. Cl. 424/70.13; 424/239.1

(57) ABSTRACT

A composition adapted for human tissue enhancement includes crystallized dextran microparticles.

### CROSS REFERENCE TO RELATED APPLICATION

This application claims the benefit of U.S. Provisional Application No. 60/563,474, filed Apr. 20, 2004.

### FIELD OF THE INVENTION

The present invention relates generally to biocompatible implants and methods for tissue enhancement.

### BACKGROUND OF THE INVENTION

Tissue enhancement, such as soft tissue enhancement, has been used for medical and cosmetic purposes. The enhancement may be surgical, such as plastic surgery enhancement, and non-surgical, such as injection of a biomaterial which augments and enhances the properties of the tissue. The term “enhancement” as used herein includes an increase in tissue volume (i.e., tissue augmentation) and/or an improvement in tissue function. The term “biomaterial” is a material used for therapies in the human body, such as a material which provides tissue enhancement.

For example, medical soft tissue enhancement may be used for treating facial wasting or lipoatrophy in HIV positive individuals. Facial lipoatrophy refers to subcutaneous fat loss in the cheeks and temples resulting in a bony, emaciated appearance. The condition may be mild to severe. As with other symptoms of lipodystrophy, or body fat abnormality syndrome (such as fat loss in the limbs and buttocks), the only thing known for certain about facial wasting is that it exists. Precise causes have not been identified and successful strategies to prevent the condition remain elusive. A recently developed cosmetic treatment for facial wasting, polylactic acid (PLA) microspheres (marketed under the trade name New-Fill®), appears to be well tolerated in European clinical trials and anecdotal reports. Although the treatment has been approved in Europe and Mexico, the future of New-Fill access in the U.S. remains uncertain.

Cosmetic soft tissue enhancement has been used to augment the volume of cheeks, lips, breasts, buttocks and legs as well as to reduce wrinkles and folds in the skin. Other cosmetic soft tissue enhancement has been used for rhinoplasty (nose reshaping).

Recently, a soft tissue enhancement composition containing dextran microspheres and hyaluronic acid (also known as hylan gel) has been developed under the trade name Reviderm Intra® by Rofil Medical International of the Netherlands. This composition is used to reduce wrinkles and folds in the skin. As provided on the Rofil website, www.rofil.com, Reviderm Intra® contains the following composition for 1 ml per syringe: 20 mg of hyaluronic acid, 25 mg of dextran microspheres, 9 mg of sodium chloride, 1 mg of phosphate buffer and sterile water added up to 1 ml. The microspheres are dispersed in the stabilized, cross-linked pharmaceutical grade hyaluronic acid gel. The microspheres are completely round and have a size of 40 to 60 microns. From photos of the dextran microspheres on the www.rofil.com website, it appears that these microspheres are cross-linked, swellable microspheres.

The dextran microspheres function to stimulate new tissue growth at the site of the injection to correct the wrinkle. In other words, the microspheres initiate collagen synthesis, in turn building a new collagen network, while replenishing hyaluronic acid depots in the soft tissue. The hyaluronic acid restores the natural fluid balance in the skin to enhance wrinkle removal.

The www.rofil.com website recommends that Reviderm Intra® be injected several times in small amounts during each treatment session. Since the dextran microspheres and hyaluronic acid are biodegradable, the filler lasts 12-24 months. Thus, Reviderm Intra®) requires repeated applications and has a limited duration as soft tissue filler.

### BRIEF SUMMARY OF THE INVENTION

One preferred aspect of the present invention provides a composition adapted for human tissue enhancement comprising crystallized dextran microparticles.

Another preferred aspect of the present invention provides a method of human tissue enhancement comprising introducing a composition comprising crystallized dextran microparticles into the human tissue to enhance the human tissue.

### BRIEF DESCRIPTION OF THE DRAWINGS

FIG. 1 is a photograph of crystallized dextran microparticles spontaneously formed in 55.0% (W/W) aqueous solution of dextran with MW 40.0 kDa.

FIG. 2A is a photograph of a cross-section of crystallized dextran microparticles shown in FIG. 1.

FIG. 2B is a photograph of a cross-section of a microparticle shown in FIG. 2A. Microporous structure of the microparticle can be seen.

FIG. 3 is a photograph of aggregates of crystallized dextran microparticles.

FIG. 4 is a photograph of a subcutaneously injected implant consisting of crystallized dextran microparticles shown by FIG. 3.

FIG. 5 is a photograph of an intramuscular injected implant consisting of crystallized dextran microparticles shown by FIG. 3.

FIGS. 6A, 6B and 6C are photographs of a cross-section of mouse muscle with injected implant consisting of crystallized dextran microparticles (1^{st}, 4^{th}, and 28^{th} day after injection, respectively).

FIGS. 7A and 7B are photographs of a cross-section of mouse muscle with injected implant consisting of crystallized dextran microparticles (180 days after injection).

FIGS. 8A, 8B, 8C, 8D, 8E, 8F and 8G are photographs of a cross-section of mouse skin with injected implant consisting of crystallized dextran microparticles (1^{st} day, 4^{th} day, 28^{th} day, 180 days, 180 days and 1 year after injection, respectively).

FIG. 9 is a photograph of a slow release of the fluorescently labeled macromolecules from the implant which includes crystallized dextran microparticles into mouse muscle tissue on the 14^{th} day after intramuscular injection.

FIG. 10 is a photograph of an expression of the reporter gene in a mouse's muscle tissue following the plasmid DNA release from the implant.

FIG. 11 is a photograph of an emulsion of aqueous solution of PEG in aqueous solution of dextran (MW 500 kDa) containing crystallized dextran microparticles shown in FIG. 1.

FIG. 12 is a photograph of an emulsion of aqueous solution of dextran (MW 500 kDa) containing crystallized dextran microparticles shown in FIG. 1 in aqueous solution of PEG.

FIG. 13 is a photograph of an intramuscular injection of emulsion of aqueous solution of PEG in aqueous solution of dextran (MW 500 kDa) containing crystallized dextran microparticles shown in FIG. 1.

FIG. 14 is a photograph of a subcutaneous injection of emulsion of aqueous solution of PEG in aqueous solution of dextran (MW 500 kDa) containing crystallized dextran microparticles shown in FIG. 1.

FIGS. 15A and 15C schematically illustrate partition behavior of different types of particles and phases in an aqueous two phase system.

FIG. 15B is a photograph of a cross section of an implant structure based on the two phase system.

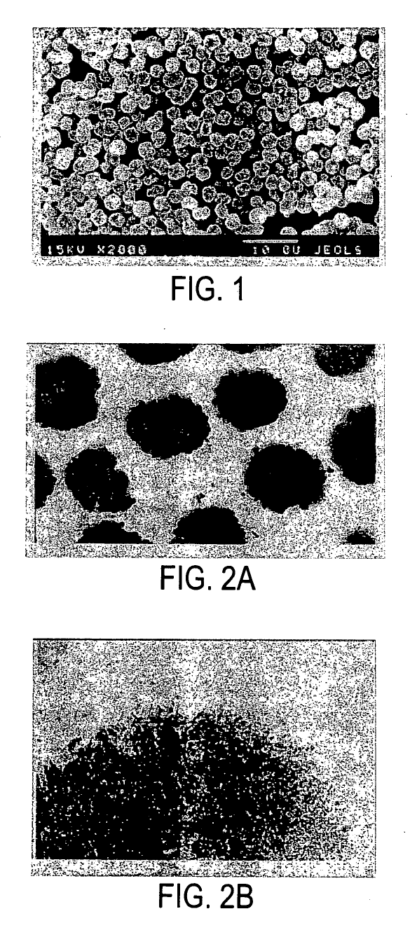

### DETAILED DESCRIPTION OF THE PREFERRED EMBODIMENTS

The present invention will now be described more fully hereinafter with reference to the accompanying drawings, in which embodiments of the invention are shown. The invention may, however, be embodied in many different forms and should not be construed as limited to the embodiments set forth herein. Rather, these embodiments are provided so this disclosure will be thorough and complete and will fully convey the scope of the invention to those skilled in the art. Like numbers refer to like elements throughout.

The present inventor has realized that a composition comprising crystallized dextran microparticles may be used to enhance tissue, such as soft tissue. The terms “enhance” and “enhancement”, as they relate to tissue, include tissue augmentation (i.e., an increase in tissue volume) and/or an improvement in tissue function. The crystallized dextran microparticles have many advantages for soft tissue enhancement. Their dissolution products are readily removed from the body by normal physiological routes. The dextran microparticles are non-toxic, non-immunogenic, biocompatible, biodegradable and do not contain animal product. The microparticle formation process does not require the use of organic solvents which may be harmful to humans if retained in the microparticles. No chemical cross-linking is involved in crystallized dextran microparticles. This results in porous microparticles, whose porous structure may be used for diffusion and adsorption of biomolecules. Optionally, a tissue enhancement agent, e.g. growth factor, may be located in the pores of the dextran microparticles for controlled release from the pores over time. This increases the duration of tissue enhancement. Furthermore, the surface characteristics of crystallized dextran microparticles may be easily optimized for anchorage dependent cells.

The term tissue enhancement includes any suitable enhancement technique desired for any suitable tissue. Preferably, the tissue is human soft tissue. The term enhancement includes, but is not limited to increasing the tissue volume, removing or decreasing wrinkles and/or folds in the skin, treating facial wasting or lipoatrophy and providing filler material into tissue that was subjected to plastic surgery. Thus, the term tissue enhancement includes surgical as well as non-surgical or cosmetic enhancement. The composition containing crystallized dextran microparticles may be used to repair cosmetic defects, congenital anomalies and/or acquired defects in the tissue.

The composition preferably comprises a suspension which contains a biocompatible fluid carrier, such as a solvent, in which the dextran microparticles are located. Preferably, the fluid carrier comprises water, such as sterile water. However, PBS (phosphate buffer saline) and suitable organic solvents such as alcohol based solvents may also be used in combination with water. The fluid carrier may comprise 1 to 99 volume percent of the composition, while the microparticles and the therapeutic agent, if any, may comprise 99 to 1 volume percent of the composition.

Preferably, the tissue enhancement composition contains the porous crystallized dextran microparticles which act as a tissue enhancement agent by forming an implant in the tissue. The enhancement composition may be used as a filler (i.e., to increase the volume of the tissue) and to stimulate new tissue growth at the site of the injection.

Alternatively, the tissue enhancement composition comprises porous, crystallized dextran microparticles and an additional tissue enhancement agent. Most preferably, at least a portion of the tissue enhancement agent is located in the pores in the dextran microparticles. Thus, the composition is used as a controlled release system. Some of the tissue enhancement agent may be located on the surface of the dextran microparticles and/or between the dextran microparticles.

In one embodiment, the controlled release tissue enhancement agent comprises botulinum toxin, such as botulinum toxin types A (for example such as toxin sold under the trade name BOTOX® in a composition including human serum albumin and NaCl), B, F, C₁, D, E, and/or G. The botulinum toxin containing composition may be injected intramuscularly to treat brow furrows (glabellar lines), to treat constipation by intersphincter injection of the puborectalis muscle, to treat blepharospasm by injection into muscles of the eyelids, and to treat upper limb spasticity, as described in U.S. Pat. No. 6,585,993, incorporated by reference in its entirety. The composition may contain from 5 to 200 units of botulinum toxin, depending on the end use, as described in U.S. Pat. No. 6,585,993. For example, 5 to 10 units of the toxin are used to treat furrowed brows.

The use of porous crystallized dextran microparticles with botulinum toxin is advantageous because a lower amount of the toxin may be used compared to compositions that do not contain the dextran microparticles and because the highly toxic toxin may be controllably released from the pores over a long time. Thus, the tissue is not exposed to a large amount of toxin at once after the injection of the composition into the skin. This improves the safety of the toxin injection as well as the duration of the treatment.

Optionally, the microparticles may further comprise cell adhesion promoters or cells on at least a portion of their surfaces. The cells are preferably autologous cells from the subject. Most preferably, the cells are autologous cells from the same type of tissues being treated, such as fat cells, muscle cells, dermal cells, and epidermal cells. “Cell adhesion promoter” refers to any compound that, because of its presence in or association with the microparticles, promotes or enhances the adhesiveness of cells to the surface of the microparticles. These compounds are often proteins that are bound to the surface of the microparticles through covalent bonds or through adhesion of the proteins and the dextran.

The composition may also optionally comprise a therapeutic or prophylactic agent, radio-pacifying agent, contrast agent or other detectable substances, targeting agent, or mixtures thereof, providing therapeutic and other benefits to the tissue in addition to enhancement. “Therapeutic agent” refers to any substance that provides therapeutic effects to the whole organism improving its biological or physiological properties and/or treats or cures one or more diseases in mammals. An example of therapeutic agent is an anti-inflammation agent that prevents or reduces the effect of inflammation or an anti-bacterial, anti-viral, or anti-histamine agent.

In one preferred embodiment, the tissue enhancement composition comprising the porous crystallized dextran microparticles is packaged for tissue enhancement. In one aspect of this embodiment, the composition is located in a vessel in an amount dosed for administration to a human. The vessel may comprise any container which may hold the composition in a suspension, gel or solid form. The vessel may comprise for example, a plastic or glass bottle, a tube, a dropper, a pouch and/or other suitable vessels. The most preferred type of vessel is a prefilled syringe because of convenience and ease of handling.

In another preferred aspect, the vessel contains an instruction for administration of the composition to a human, such as by injection. The instruction may be printed on the vessel, such being as printed directly on the vessel or on an label attached to the vessel, or enclosed with the vessel, such being printed on a sheet of paper enclosed with the vessel in a cardboard box or in a pharmacy envelope. The instructions may describe the amount of the composition administered with each dose, the frequency that the dose should be administered, the location where the dose should be administered, how to measure the dose of the composition for injection and/or any other suitable instructions for an administering health care practitioner and/or cosmetologist. Alternatively, the instructions may comprise directions for electronically or audibly accessing the dosing and administration instructions, such as a link to a website containing the instructions or a telephone number or recording where the instructions are provided audibly.

In another preferred aspect of the present invention, tissue enhancement composition containing the crystallized dextran microparticles is provided in a kit adapted for human tissue enhancement. The kit may be provided at a point of sale or it may be assembled by a health care practitioner and/or cosmetologist from separate parts prior to administering the composition. The kit comprises the composition containing the crystallized dextran microparticles, and a device for providing the crystallized dextran microparticles into the human tissue to enhance the tissue. The device may comprise any suitable device, such as a syringe and needle. Preferably, the composition is provided into the syringe from the storage vessel prior to administration of the composition.

A method of human tissue enhancement comprises introducing a composition comprising crystallized dextran microparticles into the human tissue to enhance the human tissue. Preferably, the composition is introduced into human soft tissue such as dermal tissue. If desired, the composition may further comprise an additional soft tissue enhancement agent which is controllably released from the microparticle pores over time. Tissue enhancement refers to any change of the natural state of a tissue, such as skin and related areas due to external acts. For example, the areas that may be changed by dermal tissue enhancement include, but not limited to, epidermis, dermis, subcutaneous layer, fat, arrector pill muscle, hair shaft, sweat pore, and sebaceous gland.

A preferred method of administration is injecting the composition into an area of the subject that is in need of tissue enhancement. The injection can be carried out by syringe, catheter, needle and other means for injecting or infusing microparticles in a liquid medium. In a preferred embodiment, the injection of the injectable composition to the subject is carried out by injecting the composition into an area of the subject in need of tissue enhancement. The injection can be performed on any area of the subject's body that is in need of treatment, including, but not limited to, face, neck, torso, arms, hands, legs, and feet. The injection can be into any position in the specific area such as epidermis, dermis, fat, muscular or subcutaneous layer. The composition may also be injected extra-corporeally into organs, components of organs, or tissues prior to their inclusion into the subject's body, organs, or components of organs. The frequency and the amount of injection are determined based on the nature and location of the particular tissue deficiency being treated.

The dermal tissue enhancement method is suitable for the treatment of skin contour deficiencies, which are often caused by aging, environmental exposure, weight loss, child bearing, injury, surgery, or diseases such as HIV induced lipoatrophy, acne and cancer. The contour deficiencies include, but are not limited to frown lines, worry lines, wrinkles, crow's feet, marionette lines, stretch marks, and internal and external scars resulted from injury, wound, bite, surgery, disease or accident.

In one preferred aspect, the soft tissue, such as lip, breast, buttock, cheek and leg tissue, for example, is enhanced by increasing the tissue volume. Thus, a method of increasing a volume of human soft tissue includes providing an effective amount of a soft tissue filler composition comprising crystallized dextran microparticles into the human soft tissue to increase the volume of the soft tissue.

In another preferred aspect, the composition containing crystallized dextran microparticles and optionally the soft tissue enhancement agent is introduced into human dermal tissue, such as the epidermis, dermis, fat, subcutaneous layer, or muscular to enhance the soft tissue by removing or decreasing at least one of wrinkles and folds in the dermal tissue. Thus, a method of removing or decreasing at least one of wrinkles and folds in human dermal tissue includes injecting an effective amount of a soft tissue enhancement composition comprising crystallized dextran microparticles into the human dermal tissue containing at least one of wrinkles and folds to remove or reduce the at least one of wrinkles and folds.

In another preferred aspect, the composition containing the crystallized dextran microparticles is introduced into the human soft tissue subjected to plastic surgery as a filler after the plastic surgery. The composition may contain the fluid carrier and is introduced by injection.

In another preferred aspect, the composition containing the crystallized dextran microparticles is introduced into the human soft tissue to treat lipoatrophy. Thus, a method of treating lipoatrophy in a human includes introducing a composition comprising an effective amount of crystallized dextran microparticles into a soft tissue region of a human affected by the lipoatrophy.

While the above description focused on enhancing soft tissue in humans, the method and composition of the present invention should not be considered so limited. The method and composition described above may also be used to enhance tissue of mammals other than humans as well as to treat various medical conditions and ailments associated with mammal tissue.

The dextran microparticles may be made by any suitable crystallization process that does not involve intentional cross linking. Preferably, but not necessarily, the microparticles are formed in an aqueous solution without using an organic solvent. A method to manufacture non cross-linked, porous crystallized dextran microparticles includes preparation of a dextran solution, such as an aqueous dextran solution, conducting a crystallization process to form crystallized porous dextran microparticles, and if desired, isolating crystallized porous dextran microparticles from the solution.

If the optional, additional tissue enhancement agent is present in the composition, then the crystallized dextran microparticles and the tissue enhancement agent are combined in water after the microparticles have been crystallized to form an aqueous suspension. The enhancement agent is permeated into the pores of the microparticles by providing the enhancement agent into the solution before crystallization or by providing the isolated microparticles and the enhancement agent into a second solution, such as a second aqueous solution. The microparticles may be added to the solvent, such as water, before, at the same time and/or after adding the agent to the solvent. For example, the porous microparticles may be formed first and then the enhancement agent is provided into a solution containing the microparticles to allow the enhancement agent to permeate into the pores of the microparticles. Of course, some of the enhancement agent may also become attached to the surface of the microparticle in this process.

If the additional enhancement agent is present in the composition, then the microparticles preferably have sufficient porosity to contain the enhancement agent within the pores and to provide a timed release of the enhancement agent from the pores. In other words, the enhancement agent is released over time from the pores, such as in over one hour, such as in several hours to several months, rather than all at once. Thus, the particle material, pore size and pore volume can be selected based on the type of enhancement agent used, the quantity of enhancement agent needed for delivery, the duration of the delivery of the enhancement agent, the environment where the enhancement agent will be delivered and other factors. Preferably, the enhancement agent is not encapsulated in the microparticle (i.e., the microparticle does not act as a shell with the enhancement agent core inside the shell). However, if desired, a portion of the enhancement agent may also be encapsulated in a microparticle shell and/or is attached to the surface of the microparticle in addition to being located in the pores of the microparticle. The location of the enhancement agent in the pores provides an optimum timed release of the enhancement agent. In contrast, the enhancement agent attached to the surface of the microparticle is often released too quickly, while the enhancement agent encapsulated in the microparticle is often not released soon enough and is then released all at once as the microparticle shell disintegrates.

The microparticles may be administered to a mammal in the solvent in which they were formed. Alternatively, they may be removed from the solvent in which they were formed and placed into water or other aqueous solutions for administration, or dried and provided in xerogel or powder form.

The following specific examples should not be considered limiting on the scope of the present invention.

The present inventor has experimentally found that crystallized dextran microparticles with an average diameter ranging from 0.5 to 3.5 microns were spontaneously formed in concentrated aqueous solutions of dextrans (10-90% W/W) with molecular weights ranging from 1.0 to 200.0 kDa, at temperature ranging from 0-99.9° C. Aqueous solution of the dextran with molecular weight of 40 kDa, 50% W/W and 60° C. is preferable for preparation of crystallized dextran microparticles. The microparticles may have any suitable shape such as a regular or an irregular shape, but are preferably spherical in shape, and are preferably 10 microns in diameter or less, such as 0.5 to 5 microns.

Transmission Electron Microscopy revealed the microporous structure of the crystallized dextran microparticles (see FIGS. 2A, 2B). Preferably, the microparticle porosity is at least 10 percent by volume, such as about 10 to about 50 percent, more preferably about 20 to about 40 percent. Thus, the structure comprises microporous microparticles with areas of macroporosity located between the particles in concentrated suspensions or xerogels.

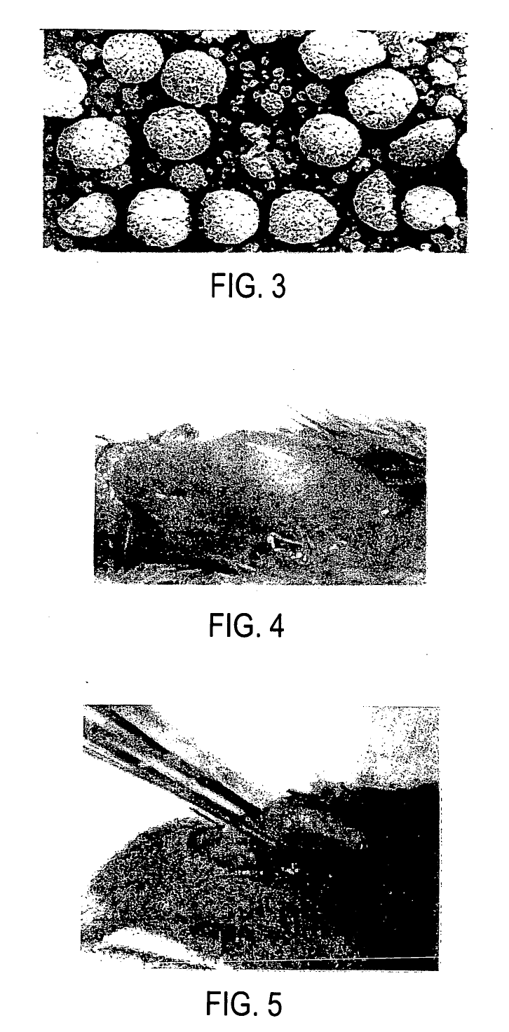

Spray drying of aqueous suspensions of the crystallized dextran microparticles has shown the possibility to produce substantially spherical aggregates of crystallized dextran microparticles with a diameter ranging from 10.0 to 150.0 microns (see FIG. 3).

A non limiting example of a method of forming the dextran microparticles is as follows. 50.0 g of dextran T40 (40 kDa molecular weight) from Amersham Biosciences is added to 50.0 g of sterile distilled water in a 500 ml lab beaker to obtain 50% w/w solution. The mixture is stirred at 60° C. (e.g. water bath) on a magnetic stirrer at 50 rpm until the dextran is completely dissolved and a clear solution is obtained. The solution may be vacuumed to remove all air inclusions. The clear solution is placed in lab oven at 60° C., for example under a lid, such as under a Tyvek® lid. 3.5 hours later, a turbid viscous suspension is developed as a result of formation of crystallized dextran microparticles.

To eliminate non-crystallized dextran, the microparticles are washed by centrifugation, for example 3,000 g, 30 min, with 3×250 ml of distilled sterile water, or by filtration of diluted suspension of microparticles, for example one part microparticles and 10 parts water (3×250 ml of distilled sterile water through sterilization filter). The microparticles are placed in 500 ml lab beaker and dried at 60° C. in lab oven for 8-12 hours to reach a moisture level of about 3-5%. The resulting dry powder consists of particles with a mean diameter of about 2 microns.

Concentrated suspensions of the crystallized dextran microparticles or aggregates thereof were tried as implants in experiments with mice to test their biocompatibility following injection into the animal's body.

FIGS. 4 and 5 show the implant in tissue following subcutaneous (FIG. 4) and intramuscular (FIG. 5) injections in experimental animals (mice). No inflammation reactions were detected in the animal's tissue during 180 days.

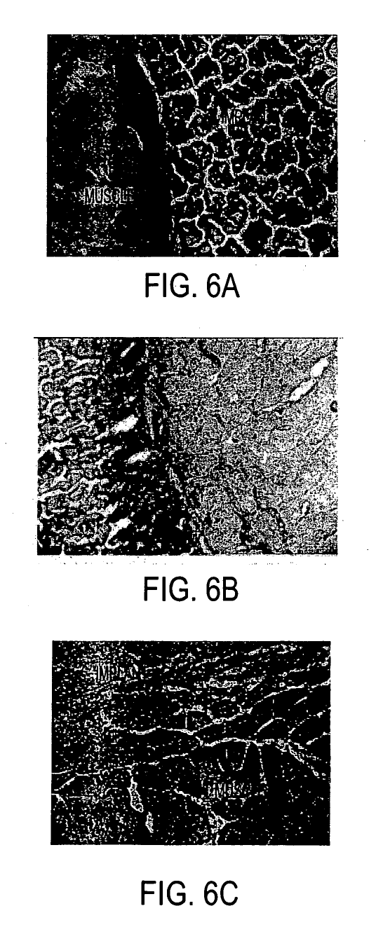

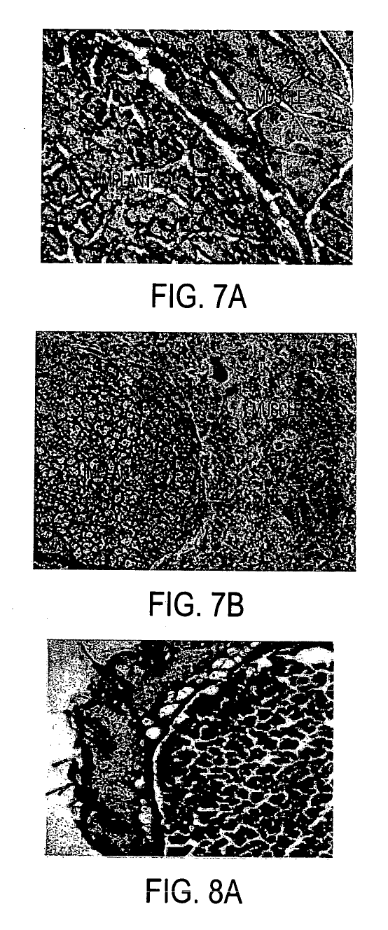

FIGS. 6A, 6B and 6C show the implant in muscle tissue of a mouse 1, 4 and 28 days, respectively, after the injection. FIGS. 7A and 7B both show the implant in muscle tissue of a mouse 180 days after the injection.

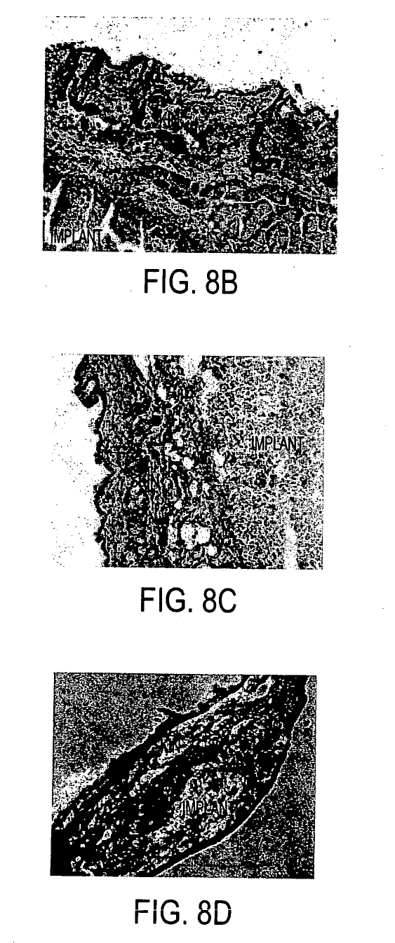

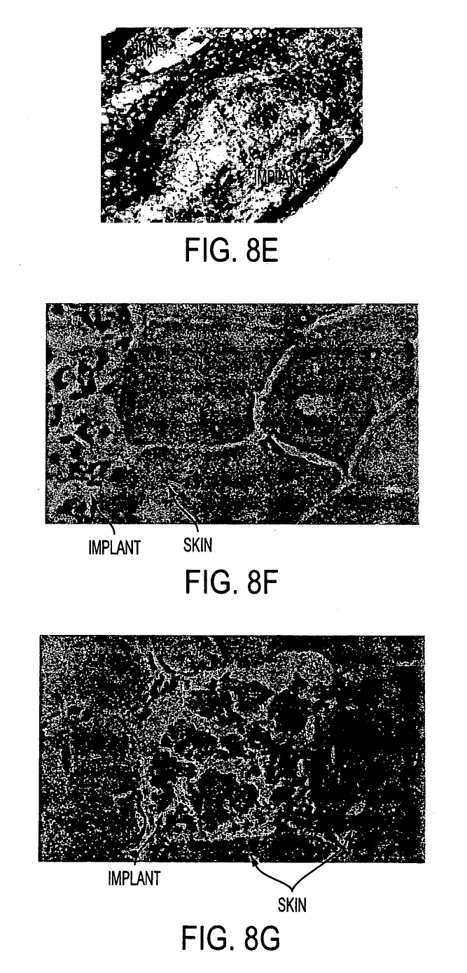

FIGS. 8A, 8B and 8C show the implant on the 4th, 11th and 28th day, respectively, after the subcutaneous injection. FIGS. 8D and 8E both show the implant 180 days after the subcutaneous injection. FIGS. 8F and 8G both show the implant one year after the subcutaneous injection. As shown in FIG. 8G, normal tissue rather than scar tissue forms at the implant site. Thus, the implant remains in the human soft tissue for at least one year without complete degradation and substantially without adverse effect.

The slow release of macromolecules from implants has been demonstrated in experiments where macromolecules were dissolved in aqueous suspensions of crystallized dextran microparticles or their aggregates before injections.

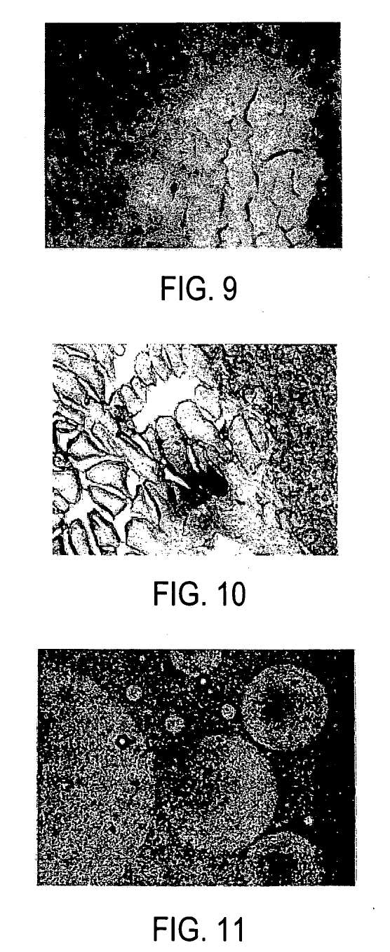

FIGS. 9 and 10 show the implant containing fluorescently labeled macromolecules (FITC-dextran, MW 500 kDa) and slow release of the macromolecules from the implant into a mouse muscle tissue on the 14^{th} day after the intramuscular injection (FIG. 9) and expression of the reporter gene in a mouse's muscle tissue following the plasmid DNA release from the implant (FIG. 10).

Self assembled structures of implants based on crystallized dextran microparticles and their aggregates may be formed based on two phase systems.

Colloidal systems such as droplets of oil, liposomes, micro- and nano-particles and cells can be dispersed in a suspension of crystallized dextran microparticles and injected to form an implant releasing therapeutic agent(s) following administration into the mammal body.

For example, in the case of oil, a special kind of implant structure can be formed where the oil core is surrounded with a shell composed of crystallized dextran microparticles or aggregates thereof dispersed in water or aqueous solutions of organic polymers such as polysaccharides (e.g. dextrans). The structure described can be designated as a capsule. It should be noted that the shell may comprise a roughly spherical shaped shell which results when the capsule is surrounded by tissue. However, when the capsule is located near a barrier, such as a bone, the capsule may comprise a core located between one or more walls of microparticles on one side and the barrier on the other side. Furthermore, while oil is used as an illustrative example, the core may comprise other materials, such as other polymers, cells, etc.

To form the capsule structure, two-phase aqueous systems are applied. When aqueous solutions of different polymers are mixed above certain concentrations they frequently form immiscible-liquid two-phase solutions. Each of the phases usually consists of more than 90% water and can be buffered and made isotonic. If a particle suspension is added to such a system, the particles are frequently found to have partitioned unequally between phases. This preferential partition behavior can be used as a basis for separation procedures for differing particles since partition in these systems is determined directly by particle surface properties. Particles which do not have identical surface properties exhibit sufficiently different partition behavior.

The competitive phase absorption of particles in the two phase system depends on the chemical nature of the polymers. A two-phase polymer method has been applied to separate or partition cells, proteins, nucleic acids and minerals (“Partitioning in Aqueous Two-Phase Systems”, 1985, eds., H. Walter, D. Brooks, and D. Fisher, pubis. Academic Press).

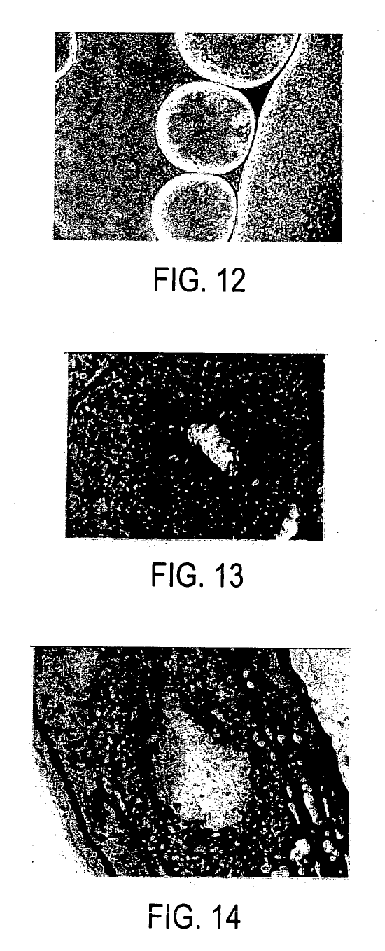

The experiments with the distribution of crystallized dextran microparticles in phase systems derived from, for instance, dextran/polyethylene glycol (PEG) mixtures, revealed that the dextran microparticles prefer to be in the dextran phase, while another PEG phase can be dispersed in this dextran phase to form a W/W emulsion and vice versa in the case when the volume of the PEG phase is bigger than the volume of the dextran phase, as shown in FIGS. 11 and 12.

FIG. 11 is a photograph of an emulsion of aqueous solution of PEG in aqueous solution of dextran containing crystallized dextran microparticles. In the structure of FIG. 11, the volume of the PEG phase is less than the volume of the dextran phase. The dextran phase contains the dextran and the crystallized dextran microparticles. Thus, the PEG phase forms into one or more sphere shaped cores surrounded by dextran/dextran microparticle shells (i.e., a closed pore structure).

FIG. 12 is a photograph of an emulsion of aqueous solution of dextran containing crystallized dextran microparticles in aqueous solution of PEG, where the volume of the PEG phase is greater than the volume of the dextran phase. In this case, the dextran phase forms into one or more spheres containing the dextran microparticles surrounded by a PEG phase (i.e., an open pore structure implant that is formed in vivo while PEG dissipates in tissue liquid). As can be seen in FIG. 12, the smaller volume (droplet) dextran phase forms into a large spherical dextran/dextran microparticle core (bottom right of FIG. 12) to which smaller spheres comprising dextran/dextran microparticles are joining and fuse with.

Thus, when the ratio of the volume of the first phase (such as the PEG phase and its inclusions, such as a therapeutic and/or enhancement agent or microparticles containing or cells producing thereof) to the volume of the second phase (such as the dextran phase and its inclusions, such as the dextran microparticles) is less than one, then the capsule forms by self assembly with a first phase core surrounded by a second phase shell. If the composition contains a therapeutic or enhancement agent which prefers to partition into the PEG phase, and the dextran microparticles which prefer to partition into the dextran phase, then the therapeutic or enhancement agent selectively partitions into the PEG core while the microparticles selectively partition into and form the shell around the PEG core by self assembly.

The emulsion can be prepared by the mixing of separately prepared dextran and PEG phases and both can be suspensions of different types of particles that prefer to be in the PEG phase or in the dextran phase respectively. The principle is that the partition of particles into different polymer phases depends on their surface structure and interfacial energy of the particles in the polymer solutions.

Injection of aqueous two phase systems containing crystallized dextran microparticles into tissues of experimental animals revealed the formation of implants with the capsule structure as shown in FIGS. 13 and 14. The volume of the dextran phase is greater than the volume of the PEG phase in the two-phase system. Both FIGS. 13 and 14 show that a capsule with a PEG core and a dextran/dextran microparticle shell forms by self assembly in vivo (i.e., after injection into mammal tissue). The shell comprises macroporous regions between adjacent microparticles as well as microporous regions in the microparticles themselves.

It should be noted that the dextran microparticles may be prepared from a different molecular weight dextran than the dextran in the solution which is provided in the two phase system. Thus, the crystallized dextran microparticles may be formed in a lower molecular weight dextran solution, such as a 5 kDa solution, than the dextran solution which is provided into the two phase system, which may be a 500 kDa dextran. The lower molecular weight solutions may be used to decrease the crystallization time and increase cristallization rate. Furthermore, lower molecular weight microparticles may dissolve faster in vivo.

The capsule structure formed from a two phase system is advantageous because it allows for a more even and prolonged release of the therapeutic or enhancement agent from the core than from a composition comprising a single phase containing the microparticles. Furthermore, it is believed that by using the capsule structure, a lower amount of microparticles may be needed to achieve the same or better timed release of the agent than if a single phase system is used. Furthermore, by controlling the amount of microparticles in the two phase system, it is believed that the thickness of the microparticle shell may be controlled. A thicker shell results from a larger amount of microparticles in the two phase system. Thus, the amount, duration and/or timing of the release of the agent from the capsule core may be controlled by controlling the thickness of the shell. Therefore, the release profile of the agent may be customized for each patient or groups of patients.

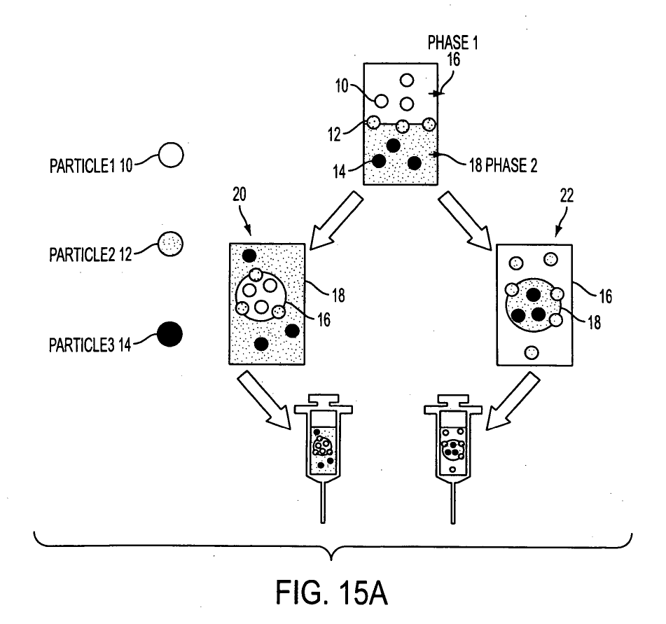

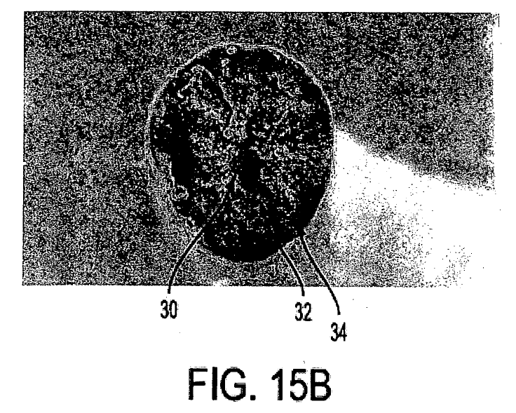

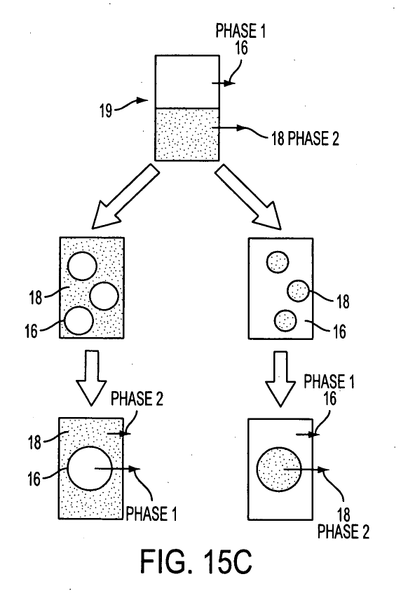

It should be noted that while PEG and dextran are used as examples of the materials of the two phases, any other suitable materials which show the following phase formation behavior may be used instead. FIG. 15A schematically illustrates partition behavior of different types of particles in an aqueous two phase system. For example, three types of molecules or molecular aggregates, which are preferably particles 10, 12 and 14, and two phases 16 and 18 are shown in FIG. 15A. However, there may be two, or more than three types of particles. The particles may be microparticles such as microspheres or nanospheres prepared from organic and/or inorganic materials, liposomes, living cells, viruses and macromolecules. The first type particles 10 preferentially segregate into the first phase 16. The second type particles 12 preferentially segregate to the boundary of the first 16 and second 18 phases. The third type particles 14 preferentially segregate into the second phase 18. Thus, by analogy to the previous non-limiting example, the first particles 10 may comprise a therapeutic or enhancement agent, the second 12 and/or the third 14 particles may comprise crystallized dextran microparticles, the first phase 16 may comprise a PEG phase and the second phase 18 may comprise a dextran phase.

If a smaller amount of the first phase 16 is provided into a larger amount of the second phase 18, as shown in area 20 of FIG. 15A, then a capsule type structure forms comprising discreet spheres of the first phase 16 containing a concentration of the first type particles 10, located in a second phase 18. The second type particles 12 may be located at the interface of the phases 16, 18 and act as a shell of the capsule. Particles 14 are dispersed in the second phase 18 and/or form a shell of the capsule.

In contrast, if a smaller amount of the second phase 18 is provided into a larger amount of the first phase 16, as shown in area 22 of FIG. 15A, then a capsule type structure forms comprising discreet spheres of the second phase 18 containing a concentration of the third type particles 14, located in a first phase 16. The second type particles 12 may be located at the interface of the phases 16, 18 and act as a shell of the capsule. Particles 10 are dispersed in the first phase 16 and/or form a shell of the capsule. The two phase systems 20 and 22 may be used as an implant, such as by being injected or surgically implanted into a mammal, such as an animal or human. Thus, the capsule forms a structured, three dimensional implant, with the core acting as a reservoir or depot for controlled release of the therapeutic or enhancement agent through the shell. In contrast, an implant with an even distribution of microparticles is an unstructured implant.

Furthermore, particles (i.e., molecular aggregates) 10, 12 and 14 may be substituted by a liquid material (e.g. oils) or macromolecules which selectively partition into one of the phases. It should be noted that while certain particles and agents selectively partition, the term “selectively partitioned” does not necessarily mean that 100 percent of the particles or agent partition into one of the phases. However, a majority of the selectively partitioned specie, preferably 80% of the partitioned specie, partitions into one of the phases.

FIG. 15B illustrates a scanning electron microscope image of a cross section of an implant structure based on the two phase system schematically illustrated in FIG. 15A. A two phase aqueous composition comprising a first dextran phase, a second PEG phase and crystallized dextran microparticles was injected into sepharose gel. This gel's composition mimics mammal tissue by stopping crystallized dextran microparticles diffusion from the injection side. The image in FIG. 15B illustrates the formation of a core-shell implant structure. The core comprises regions 30 and 32 surrounded by a shell 34. Region 30 is a void that is filled with a PEG phase region prior to cutting the gel for cross sectional SEM imaging. The PEG phase region drips out of the gel when the gel is cut during cross sectioning. Region 32 is an outer portion of the core comprising PEG droplets located in the crystallized dextran microparticles. Region 34 is the shell comprising the crystallized dextran microparticles which surrounds and holds in place the PEG containing core.

Without wishing to be bound by a particular theory, the present inventor believes that the core-shell structure shown in FIG. 15B forms by self assembly as shown schematically in FIG. 15C. While the first 16 and second 18 phases, such as aqueous solutions of different, incompatible polymers, are in a suitable storage container 19, such as in a glass beaker or vial, one phase 16 rises above the other phase 18. When the two phase composition is injected into a material which restricts free flow of the particles 16 and 18, such as mammal tissue or a substrate material, such as a gel which mimics the tissue, the composition self assembles into the core-shell structure. First, the phase that is present in the smaller volume forms into approximate spherical shapes, as shown in the middle portion of FIG. 15C. Then the spherical shapes join to form approximately spherical cores of one phase surrounded by shells of the other phase, as shown in the bottom of FIG. 15C. While a two phase system example of a multiphase system has been illustrated, the multiphase system may have more than two phases if desired.

The foregoing description of the invention has been presented for purposes of illustration and description. It is not intended to be exhaustive or to limit the invention to the precise form disclosed, and modifications and variations are possible in light of the above teachings or may be acquired from practice of the invention. The drawings and description were chosen in order to explain the principles of the invention and its practical application. It is intended that the scope of the invention be defined by the claims appended hereto, and their equivalents. All of the publications and patent applications and patents cited in this specification are herein incorporated in their entirety by reference. The transitional term “comprising” as used herein is inclusive or open-ended and does not exclude additional, unrecited elements or method steps.

I claim:

1. A composition comprising porous, crystallized dextran microparticles, wherein the composition is packaged for tissue enhancement.

2. The composition of claim 1, wherein crystallized dextran microparticles have an average diameter ranging from 0.5 to 5.0 microns.

3. The composition of claim 1, wherein the composition consists essentially of the crystallized dextran microparticles in a fluid carrier medium.

4. The composition of claim 1, wherein the composition is packaged for tissue enhancement by injection.

5. The composition of claim 1, wherein the composition is packaged for tissue enhancement to enhance the tissue by increasing the tissue volume.

6. The composition of claim 1, wherein the composition is packaged for tissue enhancement to remove or decrease at least one of wrinkles and folds in the dermal tissue.

7. The composition of claim 1, wherein the composition is packaged for tissue enhancement to treat lipoatrophy.

8. The composition of claim 1, further comprising a soft tissue enhancement agent located in the pores in the crystallized dextran microparticles.

9. The composition of claim 8, wherein the soft tissue enhancement agent comprises botulinum toxin.

10. The composition of claim 9, wherein the soft tissue enhancement agent is controllably released from the pores over time.

11. A kit adapted for human tissue enhancement, comprising:
crystallized dextran microparticles; and
a first means for providing the crystallized dextran microparticles into the human tissue to enhance the tissue.

12. The kit of claim 11, further comprising a soft tissue enhancement agent located in pores in the dextran microparticles.

13. The kit of claim 12, wherein the soft tissue enhancement agent comprises botulinum toxin.

14. The kit of claim 13, wherein the soft tissue enhancement agent is controllably released from the pores over time.

15. The kit of claim 11, wherein crystallized dextran microparticles have an average diameter ranging from 0.5 to 5.0 microns.

16. The kit of claim 11, wherein the first means is a means for providing the crystallized dextran microparticles into the human tissue to enhance the tissue by increasing the tissue volume.

17. The kit of claim 16, wherein the tissue is selected from lip, breast, buttock, cheek and leg tissue.

18. The kit of claim 11, wherein the first means is a means for providing the crystallized dextran microparticles into human dermal tissue to remove or decrease at least one of wrinkles and folds in the dermal tissue.

19. The kit of claim 11, wherein the first means is a means for injecting the crystallized dextran microparticles into the human tissue.

20. A composition for human soft tissue enhancement, comprising:
a fluid carrier; and
a second means for human soft tissue enhancement located in the fluid carrier.

21. The composition of claim 20, wherein the second means comprises porous crystallized dextran microparticles and the fluid carrier comprises water.

22. The composition of claim 20, further comprising a soft tissue enhancement agent located in pores in the dextran microparticles.

23. The composition of claim 20, wherein the second means is a means for tissue enhancement by increasing the tissue volume.

24. The composition of claim 20, wherein the second means is a means for tissue enhancement by removing or decreasing at least one of wrinkles and folds in dermal tissue.

25. The composition of claim 20, wherein the second means is a means for treating lipoatrophy.

26. A method of human tissue enhancement comprising introducing a composition comprising crystallized dextran microparticles into the human tissue to enhance the human tissue.

27. The method of claim 26, further comprising a soft tissue enhancement agent located in pores in the dextran microparticles which enhances soft human tissue.

28. The method of claim 27, wherein the soft tissue enhancement agent is controllably released from the pores over time.

29. The method of claim 26, wherein the composition further comprises a fluid carrier.

30. The method of claim 26, wherein the crystallized dextran microparticles are introduced into human soft tissue.

31. The method of claim 30, wherein the soft tissue is enhanced by increasing the tissue volume.

32. The method of claim 31, wherein the tissue is selected from lip, breast, buttock, cheek and leg tissue.

33. The method of claim 26, wherein the crystallized dextran microparticles are introduced into human dermal tissue to remove or decrease at least one of wrinkles and folds in the dermal tissue.

34. The method of claim 26, wherein the crystallized dextran microparticles are injected into the human dermal tissue.

35. The method of claim 26, wherein the crystallized dextran microparticles are introduced into the human soft tissue subjected to plastic surgery as a filler after the plastic surgery.

36. The method of claim 26, wherein the crystallized dextran microparticles are introduced into the human soft tissue to treat lipoatrophy.

37. The method of claim 26, wherein crystallized dextran microparticles have an average diameter ranging from 0.5 to 5.0 microns.

38. The method of claim 26, wherein the crystallized dextran microparticles form an implant in the human soft tissue.

39. The method of claim 38, wherein the implant remains in the human soft tissue for at least one year without complete degradation and substantially without adverse effects such that normal tissue rather than scar tissue forms at the implant site.

40. The method of claim 38, wherein the composition comprises a two phase composition which forms an implant comprising a dextran microparticle shell around a hollow core region.

41. A method of treating lipoatrophy in a human comprising introducing a composition comprising an effective amount of crystallized dextran microparticles into a soft tissue region of a human affected by the lipoatrophy.

42. The method of claim 41, wherein the composition further comprises a fluid carrier.

43. The method of claim 42, wherein the crystallized dextran microparticles form an implant in human soft tissue.

44. The method of claim 42, wherein the implant remains in the human soft tissue for at least one year without complete degradation and substantially without adverse effects.

45. The method of claim 42, wherein crystallized dextran microparticles have an average diameter ranging from 0.5 to 5.0 microns.

46. A method of removing or decreasing at least one of wrinkles and folds in human dermal tissue comprising injecting an effective amount of a soft tissue enhancement composition comprising crystallized dextran microparticles into the human dermal tissue containing at least one of wrinkles and folds to remove or reduce the at least one of wrinkles and folds.

47. The method of claim 46, wherein the composition further comprises a fluid carrier and a wrinkle and fold reducing agent located in pores in the dextran microparticles.

48. The method of claim 47, wherein the wrinkle and fold reducing agent comprises botulinum toxin A.

49. The method of claim 46, wherein the crystallized dextran microparticles form an implant in the human soft tissue.

50. The method of claim 49, wherein the implant remains in the human soft tissue for at least one year without complete degradation and substantially without adverse effects such that normal tissue rather than scar tissue forms at the implant site.

51. The method of claim 46, wherein crystallized dextran microparticles have an average diameter ranging from 0.5 to 5.0 microns.

52. A method of increasing a volume of human soft tissue comprising providing an effective amount of a soft tissue filler composition comprising crystallized dextran microparticles into the human soft tissue to increase the volume of the soft tissue.

53. The method of claim 52, wherein the composition further comprises a fluid carrier.

54. The method of claim 52, wherein the crystallized dextran microparticles form an implant in the human soft tissue.

55. The method of claim 54, wherein the implant remains in the human soft tissue for at least one year without complete degradation and substantially without adverse effects such that normal tissue rather than scar tissue forms at the implant site.

56. The method of claim 54, wherein the composition comprises a two phase composition which forms an implant comprising a dextran microparticle shell around a hollow core region.

57. The method of claim 54, wherein crystallized dextran microparticles have an average diameter ranging from 0.5 to 5.0 microns.

58. The method of claim 54, wherein the soft tissue is selected from lip, breast, buttock, cheek and leg soft tissue.

59. The method of claim 54, wherein the composition is introduced into the human soft tissue subjected to plastic surgery as a filler after the plastic surgery.
